(19) United States
(12) Patent Application Publication
(10) Pub. No.: US 2005/0002893 A1
(43) Pub. Date: Jan. 6, 2005

(54) Composition consisting of a polymer containing amino groups and an aldehyde containing at least three aldehyde groups

(76) Inventor: Helmut Goldmann, Tuttlingen (DE)

Correspondence Address: NATH & ASSOCIATES, 1030 15th STREET, NW, 6TH FLOOR, WASHINGTON, DC, 20005, US

(21) Appl. No.: 10/493,422

(22) PCT Filed: Oct. 24, 2002
(86) PCT No.: PCT/EP02/11880

(30) Foreign Application Priority Data
Oct. 24, 2001 (DE) 101 52 407.2

(51) Int. Cl. A61K 7/075; A61K 7/08

(52) U.S. Cl. 424/70.27; 514/55

(57) ABSTRACT

The invention relates to a composition of at least two, in particular two, biocompatible components which can be chemically crosslinked together, in particular for gluing biological tissue, comprising at least the following components: a) aqueous solution of at least one polymer having amino groups b) aqueous solution of at least one aldehyde having at least three aldehyde groups, where the composition is free of protein. The invention further relates to a provision of the composition for use as surgical tissue glue, and to a kit consisting of two substantially separate containers which contain components of the composition.

The invention relates to a composition of at least two, in particular two, biocompatible components which can be chemically crosslinked together, in particular for gluing biological tissue, comprising at least the following components:
a) aqueous solution of at least one polymer having amino groups
b) aqueous solution of at least one aldehyde having at least three aldehyde groups.

In surgery, mainly suture materials and clips are used to join separated portions of tissue. However, these techniques reach their limits in particular in minimally invasive surgery, which includes inter alia laparoscopy, thoracoscopy, athroscopy, heart surgery and intraluminal endoscopy. In these areas, the use of tissue adhesives and sealants is simpler, quicker and more reliable. Several patents describe synthetic and natural polymeric or macromeric systems which can be used for gluing soft tissues and for sealing air and fluid leaks in organs and blood vessels.

Fibrin adhesives commercially available on the market consist inter alia of human or/and bovine plasma proteins which represent a considerable health risk in relation to the transmission of infections. In addition, their adhesive force is often inadequate.

Compared with fibrin adhesives, hydrogels have distinctly greater cohesive and adhesive properties. Particularly suitable compositions are those which can be applied in the liquid state to the tissue and then cure within a short time through the formation of covalent bonds. The in situ curing is usually based on the crosslinking of macromeric systems and may take place by free-radical polymerization or by chemical reaction with bi- or multifunctional crosslinking reagents.

Free-radical crosslinking can be induced by sources of light or heat, and by oxidative free-radical formation with inorganic persulfates or enzymes. U.S. Pat. No. 6,156,345, Chudzik et al., U.S. Pat. No. 6,083,524, Sawhney et al. and U.S. Pat. No. 6,060,582, Hubbel et al. describe synthetic macromers with free-radical polymerizable end groups whose polymerization is initiated by irradiation with UV light in situ on the tissue. Besides synthetic polymers, it is also possible in this way to crosslink viscous solutions of collagen and collagen derivatives (U.S. Pat. No. 6,183,498 B1, Devore et al., U.S. Pat. No. 5,874,537 Kelman et al.). The technique is very elaborate and costly because of the additionally required source of light. As an alternative to UV activation, polymerization can also be induced by means of a source of heat. However, the necessary temperatures damage healthy cells in the tissue and frequently kill them. In principle, damage to healthy tissue is a problem with most free-radical polymerizations because they proceed exothermically, i.e. heat is released to the surroundings during the polymerization.

As an alternative to free-radical polymerization, macromers can also be chemically cross-linked via reactive groups. Carbonyl reactions in particular, as well as certain carboxyl reactions, have the desired properties, in terms of kinetics, to ensure rapid gelation of the components. U.S. Pat. No. 6,051,648, Rhee et al., describes synthetic polymers with N-hydroxysuccinimide activated carboxyl groups which crosslink with nucleophilic multifunctional polymers, with elimination of the N-hydroxysuccinimide. Owing to the lack of stability of the activated carboxyl groups in aqueous solution, it is necessary in this case to use preformed patches, which entails considerable disadvantages in particular in minimally invasive surgery.

Free lysine units in polypeptides and proteins form Schiff's bases through reaction with di- or polyaldehydes. Kowanko describes in U.S. Pat. No. 5,385,606 an adhesive composition consisting of human or animal protein and a di- or polyaldehyde, with the crosslinking preferably being carried out with glutaraldehyde. However, the use of glutaraldehyde is critical. Vries et al. (Abstract Book of the Second Annual Meeting of the WHS, Richmond, USA p. 51, 1992) were able to demonstrate that gelatin crosslinked with glutaraldehyde had a toxic effect on cells, which is not the case with pure gelatin.

In U.S. Pat. No. 6,156,488 by contrast, Tardy et al. describes a biocompatible tissue adhesive consisting of an aqueous collagen solution and of an aqueous polyaldehyde solution and thus avoids the use of small toxic molecules for the crosslinking. A tissue adhesive composed of oxidized dextran or starch and modified gelatin is also described by Mo et al. in J. Biomater, Sci. Polymer Edn. 2000, 11, 341-351. Dextran is present in many medical devices and is used for example as crosslinking component in oxidized form in wound dressings (Schacht et al, U.S. Pat. No. 6,132,759). The macromolecular crosslinking reagents are in this case prepared by oxidizing dextran or starch with sodium periodate. This reaction is described inter alia by Bernstein et al. (Natl. Cancer Inst. 1978, 60, 379-384) and is state of the art. The use of collagen in medicine is, by contrast, critical in relation to the risk of infection, particularly with regard to BSE and Kreutzfeldt-Jakob diseases. In addition, immune responses can be induced in the body by proteins.

Chitin is in nature a widespread linear, nitrogen-containing polysaccharide and forms the main constituent of the exoskeleton of arthropods (cockchafer wings, lobster and shrimp shells). Chitin is converted in concentrated sodium hydroxide solution into the deacetylation product chitosan which, in contrast to chitin, has free amino groups and is soluble in weakly acidic aqueous medium. The degradation behavior of pure and glutaraldehyde-treated chitosan, and the acute toxicity and the hemostatic effect of chitosan is described by Rao et al. (J. Biomed. Mater. Res. 1997, 34, 21-28). On the basis of the antimicrobial and hemostatic effect in combination with their high biocompatibility, chitosan and chitin are promising substances for medical devices. In U.S. Pat. No. 6,124,273, proteins are incorporated into chitosan sponges, and the composition is employed for external wounds. The chitosan sponges in this case release the proteins and expedite wound healing. Ono et al. describe a biological tissue adhesive composed of photocrosslinked chitosan (K. Ono, et al., J. Biomed. Mater. Res. 2000, 49, 289-295). The crosslinking takes place by irradiation with UV light. This costly and elaborate technique has, as already mentioned, not achieved practical use. In addition, the adhesive force of this adhesive is inadequate, being in the range of the fibrin adhesives.

The invention is based on the object of producing a composition which overcomes the prior art disadvantages mentioned, in particular avoids the risk of transmission of infectious diseases.

This object is achieved according to the invention by a composition having the features of claim 1. Preferred embodiments and developments of the composition of the invention are characterized in the dependent claims.

The fact that the composition of the invention is free of protein eliminates the risk of transmission of infectious diseases which is present on use of protein (e.g. collagen). This is a great advantage, especially with regard to a possible transmission of BSE pathogens to humans, compared with the protein-containing compositions described in the prior art. In addition, the risk of protein-related immune responses is also excluded with the protein-free composition of the invention.

A further advantage of the composition of the invention is that the gelation of the components takes, place spontaneously, and no additional sources of energy are required. Application is thus simplified and proceeds without harming tissues, because the healthy tissue is not adversely affected for example by excessively high heat energy.

A further advantage of the invention is that the components can be applied in aqueous medium and thus better covering of the wound area is ensured than is the case for example with preformed patches (cf., for example, U.S. Pat. No. 6,051,648).

In a particularly preferred embodiment of the composition of the invention, the aldehyde and the polymer having amino groups can be crosslinked together at body temperature. It is thus possible to avoid the abovementioned additional sources of energy, which in turn avoids tissue damage.

The polymer having amino groups is preferably derived from a biodegradable natural material. It is particularly preferred for the polymer having amino groups to be a polysaccharide, in particular a modified saccharide in which the amino groups are liberated by deacetylation. In a particularly preferred embodiment of the composition of the invention, the polymer having amio groups is an at least partially deacetylated chitin having a degree of deacetylation of from 50 to 100%, preferably 60 to 90%, in particular 70 to 80%. The deacetylation converts the acetamide groups in the chitin into amino groups. The effect of this in turn is, inter alia, that degradation in the body proceeds more slowly than with (nondeacetylated) chitin. It is particularly preferred for the polymer having amino groups to be chitosan. Chitosan has a procoagulant effect. Deacetylated chitin, especially chitosan, is preferably employed in water-soluble salt form (chloride, acetate, glutamate).

In a further embodiment of the composition of the invention, the polymer having amino groups is a synthetic polymer, in particular a polymer which undergoes renal elimination. This has the advantage that simple excretion of the polymer having amino groups is possible with the urine. The synthetic polymer is advantageously a modified polyvinyl alcohol having amino groups, preferably having a molecular weight of ≦50 000, in particular <50 000, preferably ≦20 000, in particular <20 000.

Examples of the modification of polyvinyl alcohol are the esterification of polyvinyl alcohol with amino acids, esterification with dicarboxylic acids or anhydrides linked to amide formation with polyfunctional amines, especially diamines, and formation of cyclic acetals.

The degree of modification can be set at any level and is not confined to the ends of the chains as, for example, in PEG or polyhydroxyalkanoates. Further multifunctional polymers having free hydroxy groups which are available are also polysaccharides such as dextran, cellulose, chitosan, hyaluronic acid, alginic acid, starch, agar, chitin and chondroitin sulfate.

### Examples of Modifications of Polyvinyl Alcohol (PVA)

a) Retrosynthesis of Alanine-Modified PVA

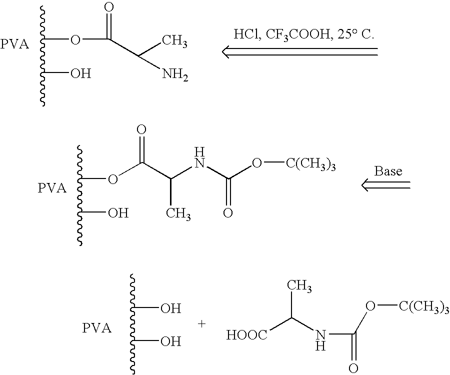

Attachment of amino acids to polyvinyl alcohol takes place in two steps. Firstly, the alcohol is esterified with a BOC-protected alanine. A base is added as catalyst. The reaction must be carried out in anhydrous solvent. After successful attachment, the BOC protective group can be eliminated under mild conditions at room temperature with trifluoroacetic acid. It is possible in principle for any desired amino acid to be attached in this way, but preference is given to amino acids having additional thiol or hydroxy groups, such as, for example, cysteine, serine, threonine, tyrosine, with particular preference for amino acids having further amino groups, such as, for example, asparagine, lysine, glutamine, arginine or trytophan. Attachment of a mixture of the amino acids mentioned is likewise conceivable.

Advantage:
no amide linkages, ester linkages ought to be cleavable by hydrolysis
degradation product is an amino acid (toxicologically acceptable)

b) Retrosynthesis with Succinic Anhydride and Diamine

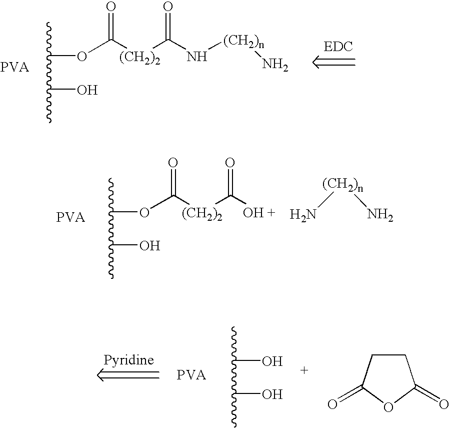

The attachment of free amino groups to polyvinyl alcohol via cyclic dianhydrides likewise takes place in two stages. In the first step, the anhydride is bound to the alcohol with the aid of a catalytically acting base EDC. This is followed by reaction with a diamine. The diamine should be employed in excess in order to avoid crosslinking of the polyvinyl alcohol during the reaction. Dianhydrides which can be used are, inter alia, also maleic anhydride, adipic anhydride or glutaric anhydride.

Advantage:
Starting substances are very favorable
Attachment to PVA through ester linkage can easily be degraded
Diamines might be toxicologically problematic (possible replacement by triethylene glycol diamine) (NH₂—C₂H₄—O—C₂H₄—O—C₂H₄—NH₂))

c) Introduction of Terminal Amino Groups via Cyclic Acetals

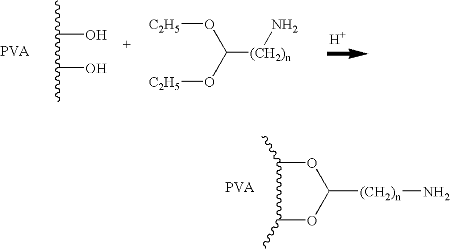

Terminal amino groups can be introduced into the polyvinyl alcohol in one step via acetal linkages. The formation of the cyclic acetal is energetically preferred in this case. The chain length of the spacer can be varied, with preference for n≦4 and particular preference for n=1.

Advantages:
Introduction of the amino group in a single synthetic step
No protective group chemistry, no cross-linking is to be expected during the reaction.

It is also possible to use combinations of polysaccharides having amino groups and polyvinyl alcohols having amino groups.

The aldehyde is advantageously a polyaldehyde. The latter is preferably of biological origin. In a preferred embodiment of the composition, the aldehyde is an oxidized polysaccharide. It is particularly preferred for both the polymer having amino groups and the aldehyde to have polysaccharide structures. In a particularly preferred embodiment of the composition, the aldehyde is an oxidized polysaccharide, the polysaccharide being at least one from the group of dextran, chitin, starch, agar, cellulose, alginic acid, glycosaminoglycans, hyaluronic acid, chondroitin sulfate and derivatives thereof. Dextranaldehyde is preferred. The aldehyde, especially the dextranaldehyde, preferably has a molecular weight of about 60 000 to 600 000, in particular about 200 000. Higher molecular weights, in particular of at least 200 000, result in high degrees of crosslinking.

The aldehyde is advantageously partially or completely masked. The purpose of the masking, especially of oxidized polyaldehydes, is to prevent the formation of intermolecular acetals and thus ensure the stability of the solutions. Controlled liberation of the aldehydes finally takes place in situ through controlled hydrolysis in a pH range from 2 to 6, preferably 2 to 4.5. It is particularly preferred for the aldehyde to be masked with an S, O or N nucleophile. It is advantageous for the partially or completely masked aldehyde to be a polysaccharide-alkali metal bisulfite adduct. In a further embodiment of the composition of the invention, the aldehyde is partially or completely masked with ethanol or glycerol.

It is advantageous for the pH values of the components to be adjusted so that the pH of a mixture of the components is between 3 and 8, in particular between 5 and 7.5. Although a high pH favors crosslinking, it leads to precipitation of, for example, chitosan.

The aldehyde in particular is responsible for the adhesive force and enables bonding to the tissue, but coverage of the tissue is impossible through the aldehyde alone. Thus, the stoichiometric amount of aldehyde groups in component b) is advantageously at least three times the stoichiometric amount of amino groups in component a).

The components are advantageously adjusted with respect to one another so that they crosslink together in a short time, in particular a time of from 15 to 200 seconds, after they are combined. The crosslinking time can be controlled for example through the concentration of the solutions and via the mixing ratio. The degree of crosslinking can likewise be adjusted, specifically via the number of aldehyde groups in the aldehyde.

The viscosity of the composition can also be controlled. The viscosities of the components are advantageously adjusted in relation to one another so that a layer of the composition with a thickness of from 0.1 to 1 mm can be applied.

The possibilities of adjustment which have been mentioned (e.g. crosslinking rate, viscosity, reactivity) do not apply to compositions with gelatin or collagen, which have been modified according to their source and do not permit defined reactions.

The content of aldehyde of component b) is advantageously from 5 to 20% by weight, in particular from 10 to 15% by weight. The content of polymer having amino groups of component a) is preferably from 1 to 25% by weight, in particular from 2 to 20% by weight. The ratios by volume of the two solutions a):b) are between 5:1 and 1:5, preferably between 3:1 and 1:3. If they are 1:1, which is preferred in many cases, it is possible in a simple manner to mix equal volumes together.

In a particularly preferred embodiment of the composition of the invention, component a) is a solution of chitosan in acetic acid, and component b) is an aqueous solution of dextranaldehyde. Dextran is distinguished for example from glutaraldehyde (cf., for example, U.S. Pat. No. 5,385,606) by being non-toxic.

The invention additionally relates to the provision of the composition of the invention for use as surgical adhesive, in particular for sealing or closing surfaces or orifices.

The components are preferably mixed together shortly before application. This can take place for example with the aid of a double-barrel syringe in which the two components are forced into a joint ejection tube in which a static mixer is present. The two components are mixed together by the static mixer in the ejection tube and are ejected, shortly before they crosslink together, from the syringe onto the application site.

A further possibility is also to mix the components only on an application site by applying the two components for example shortly one after the other to an application site.

The invention also claims a kit consisting of two containers which are substantially separate in relation to the contents, where each container in each case contains one component of the composition of the invention. In a preferred embodiment, the two containers function as syringe barrels of a double syringe. With a double syringe of this type, which is also called a double-barrel syringe, the separately stored components are forced into a joint ejection tube. The kit advantageously has a device for mixing the components. It is particularly preferred for the kit to have a static mixer which can be fitted in particular onto a double syringe. This static mixer is located in particular in the ejection tube of the double syringe. In a further embodiment, the double syringe can be closed and opened at the place where the ejection tube is fitted.

### DESCRIPTION OF THE FIGURE

FIG. 1 shows a diagrammatic longitudinal section through a preferred embodiment of the kit of the invention.

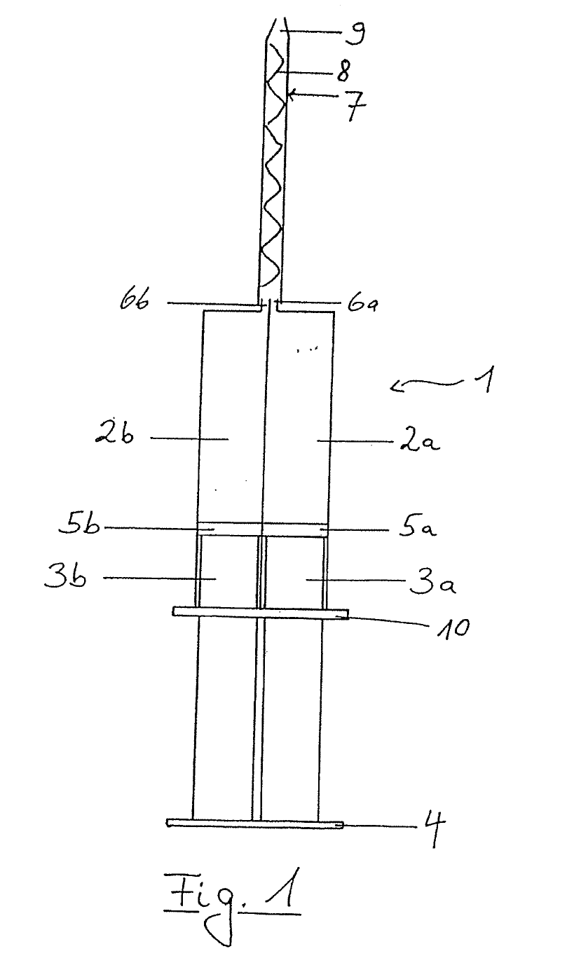

The preferred embodiment of a kit of the invention which is depicted in the single drawing shows a longitudinal section through a double syringe 1. This double syringe consists of two connected syringe barrels 2a and 2b which contain the two components of the composition of the invention separately. The ratios of the volumes of the two syringe barrels are adjusted to suit the mixing ratio of the two components. In the present exemplary embodiment, the two syringe barrels 2a and 2b have the same volumes of two components of a composition of the invention. It is also possible to use double syringes in which the volumes are different, for example the barrels have diameters of different sizes.

The double syringe 1 additionally includes two syringe plungers 3a and 3b which are connected together by a connecting plate 4. Two piston sealing rings 5a and 5b are attached at the upper end of each of the two syringe plungers 3a and 3b. These piston sealing rings are in substantially air-tight contact with the walls of the two syringe barrels 2a and 2b. At its upper end, each of the two syringe barrels 2a and 2b have in each case a mutually directly adjoining orifice 6a or 6b. These orifices are closed until the first use.

After the two adjoining orifices 6a and 6b have been opened it is possible to fit an ejection tube 7 thereon. A static mixer 8 is located in the ejection tube 7. The ejection tube narrows at its upper end to form an ejection orifice 9.

The two syringe plungers 3a and 3b with the piston sealing rings 5a and 5b affixed thereon are moved, for example by pressure on the connecting plate 4 and counter-pressure on the plate 10, in the direction of the orifices 6a and 6b. This forces the two components present in the syringe barrels out of the orifices 6a and 6b into the ejection tube 7. The two components are intimately mixed together in the ejection tube in particular by the static mixer 8 which is located in the ejection tube 7, and are finally forced in the mixed state out of the ejection orifice 9 onto an application site.

### Example of a Composition of the Invention

1. Composition

Solution A: aqueous solution of chitosan

Solution B: aqueous solution of dextranaldehyde

Mixing the two solutions results in formation of a gel which has adhesive properties. The gelation is based on the formation of imines (Schiff's bases) between the aldehyde groups in the oxidized dextran and the free amino groups in the chitosan (see reaction scheme approach 1).

As an alternative to chitosan solution, it is also possible to use solutions of modified polysaccharides (dextran modified with amines) or synthetic polymers (polyvinyl alcohol modified with amines).

1.1. Chitosan Solution

2 g of chitosan are added to 100 ml of 2% strength acetic acid solution (v/v) and stirred at room temperature for five days.

A 4% strength aqueous (w/v) Protasan® UP CI 213 (from FMC Biopolymers, Drammen, Norway) solution (deionized water) is used as alternative thereto. Protasan® UP CI 213 is a chitosan salt with chloride as counter ion.

1.2 Synthesis of Dextranaldehyde

The 5% strength (w/v) sodium periodate solution used for the synthesis is freshly prepared before each reaction and is combined with a 10% strength (w/v) dextran solution. Dextranaldehydes can be prepared by using various stoichiometric ratios (see table 1). The reaction mixture is stirred at room temperature overnight, dialyzed against distilled water for 2 days and finally the purified reaction solution is lyophilized. The reaction product is a white fibrous solid.

TABLE 1
Stoichiometric ratios of amounts in the syntheses carried out

| | | | | Proportion |
|---|---|---|---|---|
| | Molar ratio | Amount of | Amount | of oxidized |
| | NaIO₄:dextran | dextranaldehyde | of NaIO₄ | glucose units |
| Name | unit | solution | solution | (%) |
| DA 3 | 3:5 | 300 ml | 460 ml | 30 |
| | | 180 mmol | 108 mmol | |
| DA 4 | 4:5 | 300 ml | 612 ml | 33 |
| | | 180 mmol | 144 mmol | |
| DA 5 | 5:5 | 300 ml | 765 ml | 49 |
| | | 180 mmol | 180 mmol | |
| DA 6 | 2:1 | 300 ml | 1430 ml | 91 |
| | | 180 mmol | 360 mmol | |
| DA 8 | 4:1 | 150 ml | 1430 ml | 100 |
| | | 90 mmol | 360 mmol | |

15% strength solutions (w/v) of the dextran-aldehydes prepared in the manner described above were prepared by adding 4.5 g of dextranaldehyde to 30 ml of distilled water and shaking in a waterbath at 37° C. overnight. It is advantageous for the gelation to increase the pH of the dextranaldehyde solution by adding a phosphate buffer.

[image]

1.3 Dextran Molecular Weight

The average molecular weight in the dextran was varied. Dextran with an average MW of from 60 000 to 90 000 dalton (from Fischer Scientific, Schwerte, Germany) and dextran with a higher average MW of 413 263 dalton (from Sigma Aldrich Chemie GmbH, Steinheim, Germany) was employed.

The molecular weight had no effect on the proportion of oxidized glucose units as a function of the amount of NaIO₄ employed.

Determination of the Aldehyde Content

The percentage content of oxidized glucose units was determined by titrimetry in analogy to the literature [B. T. Hofreiter, B. H. Alexander, I. A. Wolff, Anal. Chem. 1955, 27, 1930 ff.].

0.15 g of dextranaldehyde is introduced into an Erlenmeyer flask and then mixed with 10 ml of a 0.25 N carbonate-free NaOH solution. The mixture is stirred until the dextranaldehyde employed is dissolved. The flask is then immersed in a hot waterbath (80° C.) for one minute and subsequently placed in an ice bath with vigorous stirring. After one minute, 15 ml of 0.25 N sulfuric acid are cautiously added while stirring. The mixture is subsequently diluted with 50 ml of water, and 1 ml of 0.2% strength phenolphthalein solution is added. The acidic solution is titrated with 0.25 N NaOH solution against the indicator.

The dialdehyde content X is calculated from the added amount of dextran or dextranaldehyde and the consumption of acid and base as follows:

$$X = [ \frac{{( {n}_{eqbase} - {n}_{eqacid} )}_{DA}}{\frac{{W}_{DA}}{161}} - \frac{{( {n}_{eqbase} - {n}_{eqacid} )}_{dextran}}{\frac{{W}_{dextran}}{162}} ] \times 100 \%$$

X: dialdehyde content
n_{eqacid}: equivalent amount of substance of the acid
n_{eqbase}: equivalent amount of substance of the base
W_{DA}: dry weight of dextranaldehyde
W_{dextran}: dry weight of dextran
n_{NaOH}: normality of the NaOH titer
n_{H2SO4}: normality of the H₂SO₄ solution used

The optimal stoichiometric ratio of NaIO₄ per glucose unit of dextran was found in further synthesis mixtures. The following graph shows that the percentage content of oxidized glucose units is above 90% when the stoichiometric ratio of NaIO₄ per glucose unit of dextran exceeds 2:1.

2. Gelation Time of the Two Solutions

The gelation time depends on the dextranaldehyde used and on the mixing ratio of the dextranaldehyde solution and the chitosan solution. The gelation time increases with an increasing degree of oxidation of the dextranaldehyde and with an increasing 15% strength dextranaldehyde solution:2% strength chitosan solution ratio. It is between 15 and 200 seconds.

TABLE 2
Gelation times as a function of the dextranaldehyde used and
of the mixing ratio of the solutions

2% strength chitosan/15%
strength dextranaldehyde
solution ratio (ml)

| Dextranaldehyde | 0.5/1.5 | 1.0/1.0 |
|---|---|---|
| DA 3 | 115 ± 31 s | 340 ± 56 s |
| DA 4 | 64 ± 10 s | 194 ± 54 s |
| DA 5 | 15 ± 2.9 s | 78 ± 33 s |
| DA 6 | 19 ± 2 s | 15 ± 2 s |

3. Determination of the Adhesive Shear Force

The adhesive shear force of the novel tissue adhesive is determined with the aid of purified and lyophilized collagen type I from bovine pericardia (Lyoplant, BBraun Aesculap, Tuttlingen). For this purpose, the Lyoplant is cut into strips 40 mm long and 10 mm wide, with the 1 cm² area to be glued being marked at the end thereof. The gluing of the Lyoplant strips proceeds as follows:

The appropriate ratios of amounts (see table) of chitosan solution and dextranaldehyde solution are combined in a test tube and shaken for 2 seconds in order to obtain thorough mixing of the solutions. Subsequently, 20 μl portions are applied centrally to the area to be glued. The glued area is protected from drying out with a film and is loaded with 50 g for 10 minutes. The strips are then drawn apart at a pulling speed of 100 mm/min. The experiments were carried out with two different batches of dextranaldehyde and the average was found for n=13 experiments. The results of the experiments are listed in table 3 to 5.

TABLE 3
Comparison of the adhesive shear force of DA 3 batch 1 and 2 as a
function of the 2% chitosan solution: 15% DA(3) solution mixing ratio

| 2% chitosan | | |
|---|---|---|
| solution/15% DA(3) | DA 3 adhesive shear | DA 3 adhesive shear |
| solution ratio by | force | force |
| volume | Batch 1 (kPa) | Batch 2 (kPa) |
| 3:1 | 121 ± 27.6 | 110 ± 27.6 |
| 1:1 | 167 ± 34.4 | 154 ± 25.7 |
| 1:3 | 137 ± 38.8 | 153 ± 33.5 |

TABLE 4
Comparison of the adhesive shear force of DA 4 batch 1 and 2 as a
function of the 2% chitosan solution: 15% DA(4) solution mixing ratio

| 2% chitosan | | |
|---|---|---|
| solution/15% DA(4) | DA 4 adhesive shear | DA 4 adhesive shear |
| solution ratio by | force | force |
| volume | Batch 1 (kPa) | Batch 2 (kPa) |
| 3:1 | 128 ± 56 | 163 ± 56.8 |
| 1:1 | 124 ± 36.4 | 175 ± 22.2 |
| 1:3 | 167 ± 54.1 | 192 ± 71.8 |

TABLE 5
Comparison of the adhesive shear force of DA 5 batch 1 and 2 as a
function of the 2% chitosan solution: 15% DA(5) solution mixing ratio

| 2% chitosan | | |
|---|---|---|
| solution/15% DA(5) | DA 5 adhesive shear | DA 5 adhesive shear |
| solution ratio by | force | force |
| volume | Batch 1 (kPa) | Batch 2 (kPa) |
| 3:1 | 136 ± 38.7 | 159 ± 37.1 |
| 1:1 | 148 ± 47.2 | 187 ± 42.9 |
| 1:3 | 223 ± 46 | 20.6 ± 41.2 |

The adhesive shear force increases just like the gelation time with increasing degree of oxidation and increasing amount of dextranaldehyde solution.

Investigations with dextranaldehyde and a polyvinyl alcohol/vinylamine graft copolymer (PVALNH₂) were carried out in analogy to the determination of the adhesive shear force of the dextranaldehyde/chitosan mixture. The graft copolymer was supplied by the manufacturer as a 20% aqueous solution and was employed in the undiluted state for gluing Lyoplant strips. The preparation of the Lyoplant strips and the application of the solutions were carried out identically to the dextranaldehyde/chitosan gluings.

The results of the investigations are listed in the tables below:

TABLE 6
Comparison of the adhesive shear force of DA 4 batch 3 as a
function of the 20% PVALNH₂ solution to 15% DA(4)
solution mixing ratio

| (20%) PVALNH₂ solution/15% DA(4) | |
|---|---|
| solution ratio by volume | Adhesive shear force (kPa) |
| 3:1 | 155 ± 25.9 |
| 1:1 | 138 ± 29.0 |
| 1:3 | 159 ± 30.6 |

TABLE 7
Comparison of the adhesive shear force of DA 5 batch 5 as a
function of the 20% PVALNH₂ solution to 15% DA(5)
solution mixing ratio

| PVALNH₂ solution/15% DA(5) | |
|---|---|
| solution ratio by volume | Adhesive shear force (kPa) |
| 3:1 | 145 ± 34.3 |
| 1:1 | 130 ± 19.0 |
| 1:3 | 198 ± 67.4 |

Additional adhesive shear force investigations were carried out with higher molecular weight dextranaldehyde and 4% Protasan solution. The solutions were applied to the Lyoplant strips with the aid of a Mixpac applicator. The applicator consists of a two-chamber system with fitted mixer tip. The Lyoplant was cut into strips with a length of 40 mm and a width of 10 mm, at the end of which a 1 cm² area to be glued was marked. The solutions were applied through the mixer, the glued area was covered with a film in order to protect it from drying out and was loaded with 50 g for 10 minutes. The strips were then drawn apart at a pulling speed of 100 mm/min. The average MW of the dextran used influences the adhesive shear force, as shown in table 8:

TABLE 8
Comparison of the adhesive shear forces of the novel adhesive
as a function of the average molecular weight of the dextran-
aldehyde. 1:1 mixing ratio of the solutions

| | | Adhesive shear |
|---|---|---|
| DA used | Chitosan solution | force [kPa] |
| DA 6 of low average MW | 4% Protasan solution | 188 ± 38 |
| (15% solution) | | |
| DA 6 of high average MW | 4% Protasan solution | 278 ± 71 |
| (15% solution) | | |

Adhesion tests were likewise carried out with Bioglue® (Cryolife International Inc. USA) consisting of proteins and glutaraldehyde, and with GLUETISS a gelatin-resorcinol dialdehyde adhesive, which were applied to the Lyoplant strips in accordance with the instructions for use. The strips glued with these adhesives were likewise covered with a film and loaded with 50 g for 10 minutes. A comparison of the adhesive shear forces achieved is shown in table 9.

TABLE 9
Comparison of the adhesive shear force of a composition of the
invention with BioGlue ® and GLUETISS ®

| | | | Adhesive |
|---|---|---|---|
| | | | shear |
| | | Mixing | force |
| Adhesive | Composition | ratio | [kPa] |
| Composition of | 4% Protosan CI 213 | 1/4 | 245 ± 68 |
| the invention | and 15% DA 6 solution | | |
| Composition of | 4% Protosan CI 213 | 1/1 | 278 ± 71 |
| the invention | and 15% DA 6 solution | | |
| Composition of | 4% Protosan CI 213 | 2/1 | 262 ± 78 |
| the invention | and 15% DA 6 solution | | |
| Bioglue | Albumin solution/ | 4/1 | 178 ± 54 |
| | glutaraldehyde solution | | |
| Gluetiss | Gelatin-resorcinol | as | 167 ± 37 |
| | solution/dialdehyde | directed | |
| | solution | | |

4. Stoppage of Liver Bleeding

The surgical glue was employed to stop bleeding in the rat liver (SPF Wistar rats). A composition of a 4% strength aqueous Protasan solution and of a 15% strength aqueous dextranaldeyde solution DA 6 was chosen for this purpose. The solutions were employed in a mixing ratio of 1:1. A Mixpac applicator was used to mix and apply the components.

After the rats had been anesthetized, the model of a crosswise incision (length of the cuts: 2.5 cm) on the liver was chosen. The adhesive was applied to the bleeding wound immediately after the incision. A gel formed and adhered firmly to the liver surface, so that the bleeding ceased immediately after application of the adhesive.

1. A composition of at least two, in particular two, biocompatible components which can be chemically crosslinked together, in particular for gluing biological tissue, comprising at least the following components:
a) aqueous solution of at least one polymer having amino groups
b) aqueous solution of at least one aldehyde having at least three aldehyde groups,
where the stoichiometric amount of aldehyde groups in component b) is at least three times the stoichiometric amount of amino groups in the polymer having amino groups, and the composition is free of protein.

2. The composition as claimed in claim 1, characterized in that the aldehyde and the polymer having amino groups can be crosslinked together at body temperature.

3. The composition as claimed in either of claims 1 or 2, characterized in that the polymer having amino groups is derived from a biodegradable natural material.

4. The composition as claimed in any of the preceding claims, characterized in that the polymer having amino groups is a polysaccharide, in particular a modified polysaccharide, in which the amino groups are liberated by deacetylation.

5. The composition as claimed in any of the preceding claims, characterized in that the polymer having amino groups is chitosan.

6. The composition as claimed in any of the preceding claims, characterized in that the polymer having amino groups is an at least partially deacetylated chitin having a degree of deacetylation of from 50 to 100%, preferably 60 to 90%, in particular 70 to 80%.

7. The composition as claimed in any of the preceding claims, in particular as claimed in claim 1 or 2, characterized in that the polymer having amino groups is a synthetic polymer, in particular a polymer which undergoes renal elimination.

8. The composition as claimed in claim 7, characterized in that the synthetic polymer is a modified polyvinyl alcohol having amino groups, preferably having a molecular weight of ≦50 000, in particular ≦20 000.

9. The composition as claimed in any of the preceding claims, characterized in that the aldehyde is a polyaldehyde.

10. The composition as claimed in any of the preceding claims, characterized in that the aldehyde is an oxidized polysaccharide.

11. The composition as claimed in claim 10, characterized in that the polysaccharide is at least one from the group of dextran, chitin, starch, agar, cellulose, alginic acid, glycosaminoglycans, hyaluronic acid, chondroitin sulfate and derivatives thereof.

12. The composition as claimed in any of the preceding claims, characterized in that the aldehyde, in particular dextranaldehyde, has a molecular weight of about 60 000 to 600 000, in particular about 200 000.

13. The composition as claimed in any of the preceding claims, characterized in that the aldehyde is partially or completely masked.

14. The composition as claimed in claim 13, characterized in that the aldehyde is masked with an S, O or N nucleophile.

15. The composition as claimed in either of claims 13 or 14, characterized in that the partially or completely masked aldehyde is a polysaccharide-alkali metal bisulfite adduct.

16. The composition as claimed in either of claims 13 or 14, characterized in that the aldehyde is partially or completely masked with ethanol or glycerol.

17. The composition as claimed in any of the preceding claims, characterized in that the content of aldehyde of component b) is from 5 to 20% by weight, in particular from 10 to 15% by weight.

18. The composition as claimed in any of the preceding claims, characterized in that the content of polymer having amino groups of component a) is from 1 to 25% by weight, in particular from 2 to 20% by weight.

19. The composition as claimed in any of the preceding claims, characterized in that the ph values of the components are adjusted so that the ph of a mixture of the components is between 3 and 8, in particular between 5 and 7.5.

20. The composition as claimed in any of the preceding claims, characterized in that the components are adjusted with respect to one another so that they crosslink together in a short time, in particular a time of from 15 to 200 sec, after they are combined.

21. The composition as claimed in any of the preceding claims, characterized in that the viscosities of the components are adjusted in relation to one another so that a layer of the composition with a thickness of from 0.1 to 1 mm can be applied.

22. The composition as claimed in any of claims 1 to 6 and 9 to 18, characterized in that component a) is a solution of chitosan in acetic acid, and component b) is an aqueous solution of dextranaldehyde.

23. The provision of the composition as claimed in any of the preceding claims for use as surgical tissue adhesive, in particular for sealing or closing surfaces or orifices.

24. The use as set forth in claim 23, characterized in that the components are mixed together shortly before application.

25. The use as set forth in claim 23, characterized in that the components are mixed on an application site.

26. A kit consisting of two substantially separate containers, where the containers each contain one component of the composition as claimed in any of claims 1 to 22.

27. The kit as claimed in claim 26, characterized in that the two containers function as syringe barrels of a double syringe.

28. The kit as claimed in either of claims 26 to 27, characterized in that it has a device for mixing the components.

29. The kit as claimed in any of claims 26 to 28, characterized in that it has a static mixer which can be fitted in particular onto a double syringe.
